(19) United States
(12) Patent Application Publication
(10) Pub. No.: US 2026/0096722 A1
(43) Pub. Date: Apr. 9, 2026

(54) FRACTIONAL TREATMENT OF CERVIX

(71) Applicant: Inmode Ltd., Yokneam (IL)

(72) Inventor: Michael Kreindel, Richmond Hill (CA)

(73) Assignee: Inmode Ltd., Yokneam (IL)

(21) Appl. No.: 18/908,835

(22) Filed: Oct. 8, 2024

(51) Int. Cl. A61B 1/303; A61B 18/00; A61B 18/14

(52) U.S. Cl. CPC A61B 1/303; A61B 18/14; A61B 2018/00577

(57) ABSTRACT

The invention relates to a device in the field of fractional ablation of cervical epithelium for the treatment of papilloma virus, cervical interepithelial neoplasia and cervix cancer.

### FIELD OF THE INVENTION

The present invention relates to fractional radio-frequency (RF) treatment of the cervix, such as the cervical epithelium.

### BACKGROUND OF THE INVENTION

Genital HPV (human papilloma virus) is the most common STI (sexually transmitted infection) in the United States for both women and men. About 79 million Americans have HPV. It is so common that 80% of women will get at least one type of HPV at some point in their lifetime. HPV disappears spontaneously for 90% of infected women during two years but impact of the infection is very strong because often it transformed to cervix intraepithelial neoplasia and then to cervix cancer which is the second most common type of cancer for women.

There are more than 200 variants of papilloma virus. High-risk HPVs can cause several types of cancer. There are about 14 high-risk HPV types including HPV 16, 18, 31, 33, 35, 39, 45, 51, 52, 56, 58, 59, 66, and 68. Two of these, HPV16 and HPV18, are responsible for most HPV-related cancers.

Cervical intraepithelial neoplasia (CIN) is a precancerous condition in which abnormal cells grow on the surface of the cervix. CIN commonly occurs after a woman becomes infected with the human papilloma virus and has a level classified by amount of cervical epithelium affected by abnormal cells.

According to consensus guideline the following treatment is recommended for cervical pre-cancerous conditions (According to Massad L S, Einstein M H, Huh W K, Katki H A, Kinney W K, Schiffman M, Solomon D, Wentzensen N, Lawson H W., 2012 ASCCP Consensus Guidelines Conference. 2012 updated consensus guidelines for the management of abnormal cervical cancer screening tests and cancer precursors. J Low Genit Tract Dis. 2013 April; 17 (5 Suppl 1): S1-S27):

CIN-1 can undergo observation and co-testing repeated in one year. If CIN-1 is persistent after two years or progresses within that time, further treatment is recommended.

CIN-2 and 3 requires treatment. The usual treatment is ablation or excision of abnormal cells. Ablation of abnormal cells includes cryosurgery or laser ablation (CO₂ laser). Ablation is only acceptable if the endocervical sampling is negative, if there are no glandular abnormalities, if the entire borders of the lesion are visible, and if the patient has not failed other treatments.

LEEP/LLETZ (loop electrosurgical excision procedure) may be recommended. Ablative procedures have a higher recurrence rate in the setting of severe dysplasia when compared to LEEP.

Excisional procedures for the treatment of CIN include LEEP, cold knife conization, and laser conization. Significant parts of the cervix are removed during the procedure, which increases the risk of miscarriage significantly.

### SUMMARY

The present invention seeks to provide a system and method for treatment of the cervix, particularly the cervical epithelium, at earlier stages to reduce risk of developing precancerous conditions and prevent CIN and cancer formation, as is described hereinbelow. Early non-excisional treatment of cervix epithelium may stop abnormal cell formation and avoid surgical intervention.

The present invention is a device and method for delivery RF energy to the surface of the cervix in uniform manner causing coagulation or ablation of a small part of the epithelium and sub-necrotic heating tissue around this area. The device includes an energy source and a hand piece delivering energy from the energy source to the cervix. The hand piece has a disposable part inserted into the vagina.

In one embodiment, the energy source is a radio-frequency (RF) generator. The RF generator has a user interface for selecting one or more of the following treatment parameters: RF power, time of energy delivery or pulse duration, RF energy, or tissue impedance range in which RF energy is delivered. Alternatively, parameters can be preset automatically by the controller and applied when the user activates the energy.

A single use disposable tip may have a mono-polar or bi-polar design. The number of electrodes in the tip is optimized to create required heating pattern. The size of the electrodes and applied RF pulse may correlate with the thickness of the epithelium to avoid damage of deeper tissue layer. One non-limiting electrode dimension is a diameter in the range of 0.1 mm to 0.5 mm, but this could be larger or smaller. The RF pulse width is preferably below 0.5 sec to prevent heat conduction to the deeper layer of the cervix.

The device measures and controls RF current, voltage, power and tissue impedance. RF power delivered to the tissue may be adjusted according to temperature measurements to reach and maintain the required tissue temperature. A temperature sensor can be embedded into the treatment hand piece at the distal end, which contacts the cervix.

In one embodiment the device has two treatment tips, one for treatment of the cervix surface and another for treatment of the cervix canal. Alternatively, the same treatment tip can be designed to treat both treatment areas simultaneously. The energy can be delivered to all electrodes simultaneously or sequentially, using electronic components for switching RF energy between the electrodes.

The treatment depth may be in the range of 0.2 mm to 0.5 mm. The electrodes delivering RF energy may be sharp to penetrate into the treated tissue or applied to the tissue surface so that RF energy and heat penetrate to the required depth.

The goal of treatment is to create prolonged inflammation and/or an immune response in the area to get rid of HPV and abnormal cells in the cervical epithelium.

The method includes applying a plurality of small electrodes to the cervical epithelium and applying energy to cause fractional thermal ablation of the tissue. Multiple treatments can be applied to reach the desired effect.

The plurality of electrodes can cover the entire treated area or only part of it. The treatment result is reached by repeated placement of the hand piece over the treatment area to reach the required density of thermal lesions and achieve the desired energy.

The RF energy used for the treatment can be, without limitation, in a frequency range from 100 kHz up to 6 MHz and RF power is varied from 1 W up to 300 W. Energy is preferably applied in pulse manner with pulse duration below 0.5 sec. Energy can be delivered with a single pulse or train of pulses. Alternatively, laser or ultrasound energy can be used to create fractional heating of the cervical epithelium.

There is thus provided in accordance with a non-limiting embodiment of the present invention a method for treatment of a cervical epithelium includes inserting a hand piece into a vaginal canal, said hand piece comprising a plurality of pin electrodes, applying said pin electrodes to the cervix surface, applying at least one pulse of radio-frequency (RF) energy to the plurality of pin electrodes, creating multiple ablation zones in a vicinity of each of said pin electrodes, each of said ablation zones being discrete zones separated from each other by non-damaged tissue.

### BRIEF DESCRIPTION OF DRAWINGS

The present invention will be understood and appreciated more fully from the following detailed description taken in conjunction with the drawings in which:

FIG. 1A is a schematic illustration of the cervix anatomy.

FIG. 1B is a schematic illustration of the cervix anatomy with the depicted treatment area.

FIG. 2 is a schematic illustration of an energy source connected to hand piece.

FIG. 3 is a schematic illustration of the hand piece applied to the treatment area.

FIG. 4 is a schematic illustration of the hand piece.

FIG. 5 is a schematic illustration of the hand piece surface for ectocervical treatment.

FIG. 6 is a schematic illustration of an alternative hand piece design for cervical treatment.

FIG. 7 is a schematic illustration of an alternative design of hand piece in which a flat element covers the entire area of the ectocervix.

### DETAILED DESCRIPTION

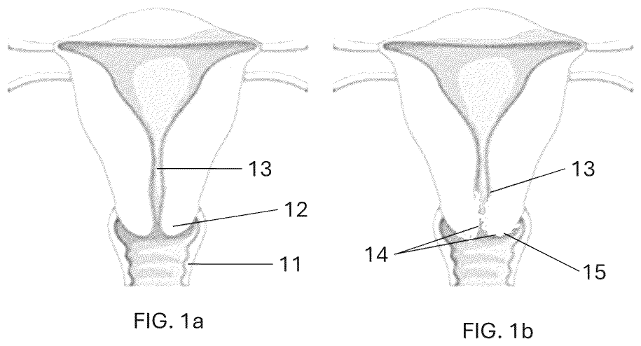

Reference is now made to FIG. 1A, which illustrates that the cervix has complex structure and difficult access for treatment. Cervix 12 located at the end of vaginal canal 11. Endocervical canal 13 is located in the center of cervix 12. Cervix size is about 1 inch in diameter and endocervical canal 13 has diameter of about 4 mm and depth about 15 mm.

FIG. 1B represents the cervix anatomy with marked surface 14 being the area usually affected by HPV and by CIN. The affected zone is different for each patient but typically affects the epithelium of ectocervix 15 and endocervical canal 13.

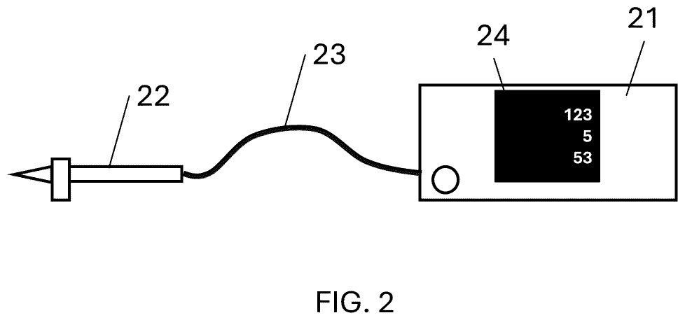

FIG. 2 shows schematically energy source 21 with user interface 24 used for setting and controlling treatment parameters. The hand piece 22 is connected to energy source 21 via harness 23. The hand piece can be sterile and disposable. Alternatively only a portion of the hand piece can be disposable.

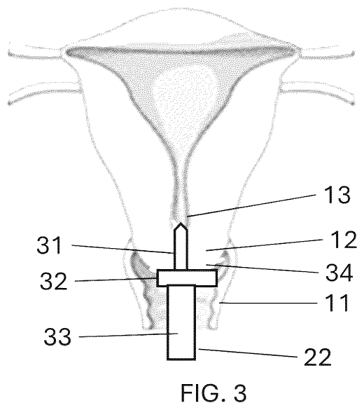

Referring to FIG. 3, the hand piece 22 is applied to the cervix 12. The hand piece 22 is inserted into the vaginal canal 11 and protruded element 31 is inserted into the endocervical canal to position hand piece 22 on the cervix 12. The contact element 32 of the hand piece 22 is applied to the epithelial layer of the ectocervix 34. The treatment attendant can rotate shaft 33 to rotate protruded element 31 and contact element 32 in order to apply energy uniformly to all treatment area. The contact surface 32 may be flat, or slightly conical or slightly curved for better contact with the tissue; other shapes are also possible.

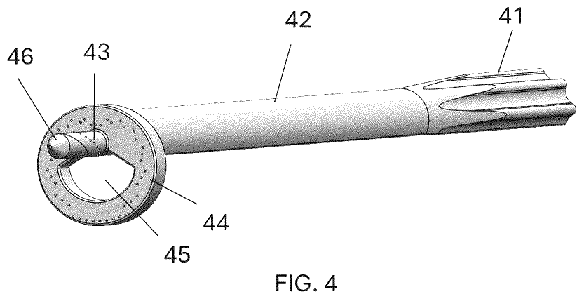

FIG. 4 shows hand piece with handle 41 and shaft 42 inserted into the vaginal canal. The distal end of hand piece has protruded element 43 with cone end 46 for inserting into the endocervical canal. The protruded element comprises small pin electrodes to deliver RF energy to the endocervical canal. A contact element 44 is designed to be attached to ectocervix. The contact element 44 is covered by pin electrodes which at rotation around hand piece axis cover the ectocervix with ablation zones uniformly. The contact element 44 has an opening 45 for observation of treatment area during hand piece insertion.

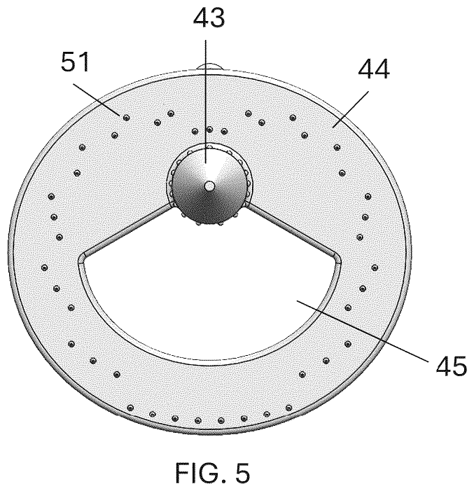

FIG. 5 shows arrangement of pin electrodes 51 over the contact element 44 around protruded element 43 and opening 45. The pins are arranged by clusters located on concentric circles. Rotation the hand piece 8 times by 45 degrees around the axis covers ectocervix with 8 circles of ablation zones uniformly distributed over the area. RF energy is delivered at each position of hand piece. Each pin electrode 51 has preferably a diameter from 0.1 mm to 0.5 mm, without limitation. Distance between ablation zone is varied from 0.1 mm up to 3 mm, without limitation.

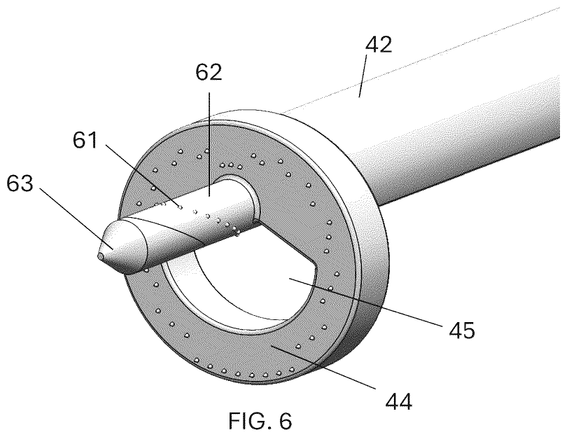

FIG. 6 shows hand piece treatment end from another angle to demonstrate pin electrodes 61 arrangement over the protruded element 62. The protruded element 62 has sharp cone end 63 for easier insertion into the endocervical canal. Rotation of hand piece 8 times by 45 degrees will cover endocervical canal with uniform pattern of ablation zone. The treatment area of endocervical canal is varied from 3 mm up to 15 mm, without limitation.

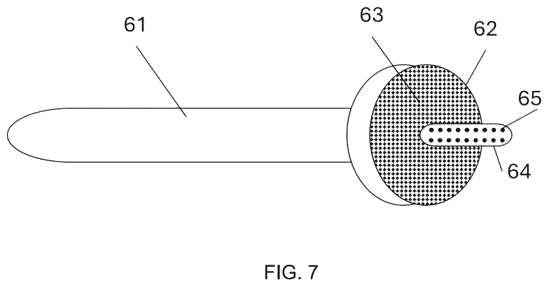

FIG. 7 shows an alternative design of hand piece where without limitation element 62 covers the entire area of the ectocervix. Pin electrodes 63 distributed uniformly over the without limitation element. The protruded element 64 is also covered by pin electrodes. RF energy is delivered to the all pin electrodes simultaneously or consequently to cover treatment are with multiple ablation zones without moving the hand piece.

The preferred parameters for device are as follows, without limitation:
Length of disposable tip is 9-20 cm
Tip treatment area is 0.5-10 cm²
Size of pin electrode is 0.1 mm to 1 mm.
Average RF power is in the range of 5 W to 400 W
Frequency of RF is from 0.1 MHz up to 6 MHz
RF energy is delivered to the pin electrodes in pulse manner. The pulse width is 1 msec to 1 sec.
RF voltage applied to the skin may be in the range of 10 V up to 1000 V

1. A method for treatment of a cervical epithelium, comprising:
inserting a hand piece into a vaginal canal, said hand piece comprising a plurality of pin electrodes;
applying said pin electrodes to the cervix surface;
applying at least one pulse of radio-frequency (RF) energy to the plurality of pin electrodes;
creating multiple ablation zones in a vicinity of each of said pin electrodes, each of said ablation zones being discrete zones separated from each other by non-damaged tissue.

2. The method according to claim 1, wherein the cervix surface is on an ectocervix and/or an endocervix.

3. The method according to claim 1, wherein the treatment is used to treat cervical interepithelial neoplasia or/and HPV.

4. The method according to claim 1, further comprising rotating said hand piece around a rotation axis to treat the cervix surface uniformly.

5. The method according to claim 1, wherein said ablation zones have a depth between 0.1 mm to 1 mm.

6. The method according to claim 1, wherein the RF energy is applied between said pin electrodes.

7. The method according to claim 1, wherein the RF energy is applied between at least one of said pin electrodes and a return electrode.

8. The method according to claim 1, wherein the RF frequency is in a range of 100 kHz to 6 MHz.

9. The method according to claim 1, wherein the RF energy has a pulse width between 1 msec and 1 sec.

10. The method according to claim 1, wherein a diameter of each of said pin electrodes is in the range of 0.1 mm to 1 mm.

11. The method according to claim 1, wherein energy per pin electrode is in a range from 10 mJ up to 1 J.
